(19) United States
(12) Patent Application Publication
(10) Pub. No.: US 2023/0033551 A1
(43) Pub. Date: Feb. 2, 2023

(54) AERIAL SAMPLING PLATFORM

(71) Applicant: Baker Hughes Holdings LLC, Houston, TX (US)

(72) Inventors: James Hyatt, Houston, TX (US); Rick Hunter, Houston, TX (US)

(21) Appl. No.: 17/854,899

(22) Filed: Jun. 30, 2022

Related U.S. Application Data
(60) Provisional application No. 63/226,567, filed on Jul. 28, 2021.

(51) Int. Cl. G01N 33/00

(52) U.S. Cl. CPC G01N 33/0073; G01N 33/0063

(57) ABSTRACT

A system is provided including an unmanned aerial vehicle. The unmanned aerial vehicle can include at least one gas sensor and a manifold coupled to the at least one gas sensor. The manifold can include at least one sample conduit including a sample inlet, a filter, and a valve coupled to the filter. The unmanned aerial vehicle can include a controller and a computing device coupled to the controller. The computing device can include a processor configured to perform operations to receive sensor data characterizing a first sample of a first gas sampled via the at least one sensor. The processor can also determine sample data associated with the first gas based on the sensor data. The sample data can include a concentration and a type of the first gas. The processor can further provide the sample data. Related methods, apparatus, techniques and articles are also described.

### CROSS-REFERENCE TO RELATED APPLICATIONS

The present application claims priority under 35 U.S.C. § 119 to U.S. Provisional Application No. 63/226,567 entitled “Aerial Sampling Platform” filed on Jul. 28, 2021, which is hereby expressly incorporated by reference in its entirety.

### BACKGROUND

Gas sampling can be performed to determine quantities or concentrations of gases at an emission site. Often gases, can be emitted from sites which are hazardous for operators seeking to collect samples of the emitted gases. The site or the emitted gases can cause harm to an operator seeking to collect the emitting gases. As well, navigating to such sites can be time-consuming and dangerous when the site is located in difficult terrain. As a result, sampling emitted gases can require extensive amounts of time to collect sufficient quantities of the gas being sampled and to return samples to a laboratory for testing to determine if a gas is being emitted at a hazardous level. It can be advantageous to reduce the amount of time to perform gas sampling in hazardous environments and to reduce operator exposure to hazard environments where a gas emission is occurring.

### SUMMARY

A system is provided including an unmanned aerial vehicle. The unmanned aerial vehicle can include at least one gas sensor and a manifold coupled to the at least one gas sensor. The manifold can include at least one sample conduit including a sample inlet, a filter, and a valve coupled to the filter. The unmanned aerial vehicle can include a controller and a computing device coupled to the controller. The computing device can include a processor configured to perform operations to receive sensor data characterizing a first sample of a first gas sampled via the at least one sensor. The processor can also determine sample data associated with the first gas based on the sensor data. The sample data can include a concentration and a type of the first gas. The processor can further provide the sample data. Related methods, apparatus, techniques and articles are also described.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is an image illustrating an exemplary embodiment of an aerial sampling platform according to subject matter described herein;

FIG. 2 is a diagram illustrating an exemplary embodiment of a sample conduit of the aerial sampling platform of FIG. 1 according to subject matter described herein;

FIG. 3 is a diagram illustrating an exemplary embodiment of a sampling manifold of the aerial sampling platform of FIG. 1 according to subject matter described herein;

FIG. 4 is a diagram illustrating an exemplary embodiment of control circuitry of the aerial sampling platform of FIG. 1 according to subject matter described herein;

FIG. 5 is an diagram illustrating an exemplary embodiment of a power adapter circuit of the aerial sampling platform of FIG. 1 according to subject matter described herein; and

FIG. 6 is a process flow diagram for determining and providing sample data of a sample collected by the aerial sampling platform of FIG. 1 according to subject matter described herein.

Like reference symbols in the various drawings indicate like elements.

### DETAILED DESCRIPTION

Sampling gases can require human operators to navigate or walk to a point of interest and to take a sample of a gas being emitted from the point of interest. In some cases, the point of interest can be associated with an industrial process or a piece of industrial machinery that can be hazardous the operator attempting to collect the gas sample. Exposure to hazardous environments can cause injury or fatality to the operator.

To mitigate risk of exposure to hazardous environments, operators can quickly enter a hazardous area and leave behind a sampling device to collect a sample of an emitted gas. Often a point of interest can be located in difficult terrain requiring extensive time for an operator to reach the point of interest and further exposing the operator to additional risk to collect a sample of a gas. Sample collecting devices can later be retrieved causing an operator to be exposed again to hazardous conditions. Additional time and specialized equipment can be required to process the retrieved sample, which can further prolong the time required to identify the gas or determine a concentration of the gas. The foregoing process can be extenuated when multiple sites must be visited by an operator to collect multiple samples from geographically separated points of interest. Thus, a need exists for more efficient gas sampling systems and methods which can reduce operator exposure to hazardous environments and provide sample data more rapidly.

The systems and methods described herein can perform gas sampling rapidly and safely by using an aerial sampling platform configured to navigate to points of interest, collect samples of gases, and provide sample data in real-time or near real-time. As a result, great numbers of points of interest can be visited in less time by flying the aerial sampling platform to each site at which a gas sample is to be collected.

The aerial sampling platform described herein includes an unmanned aerial vehicle (UAV) or drone configured with gas sensors for detecting gases such as oxygen, carbon monoxide, hydrogen sulfide, benzene, methane, and other volatile organic compounds (VOCs). The aerial sampling platform can be used to quantify gas emissions at industrial sites or during industrial operations or processes to provide more accurate data regarding the environmental impact of the gas emission at the sample site. The aerial sampling platform is not limited to hazardous industrial environments and can provide air quality monitoring, without limit, in non-hazardous environments as well.

The aerial sampling platform and methods described herein can sample multiple different gases from a single platform both in-flight and while on the ground. The aerial sampling platform can include an extendable boom to allow sampling gases away from or outside of disturbed air (e.g., prop-wash) created by rotation of the rotor blades of the UAV. In this way, collected samples are more likely to include greater quantities of the gas being sampled and reduced quantities of undesirable air or gases which is not the focus of the sampling. Thus, the extended boom can enable greater identification of gas emissions at a particular location.

In addition, the aerial sampling platform described herein can include multiple filter tubes used in association with sampling various gases. A manifold can be included in the aerial sampling platform to sequentially and programmatically collect, process and determine sample data associated with different gases. The manifold can be configured to switch sampling between different filter tubes while in flight or on the ground. The aerial sampling platform described herein can advantageously provide enhanced gas detection for multiple points of interest and can increase the number of samples collected, as well as the number of different types of gases that can be sampled. The aerial sampling platform described herein can include sensors and computing devices configured to sense and determine gases and concentrations of the gases in real-time or near real-time.

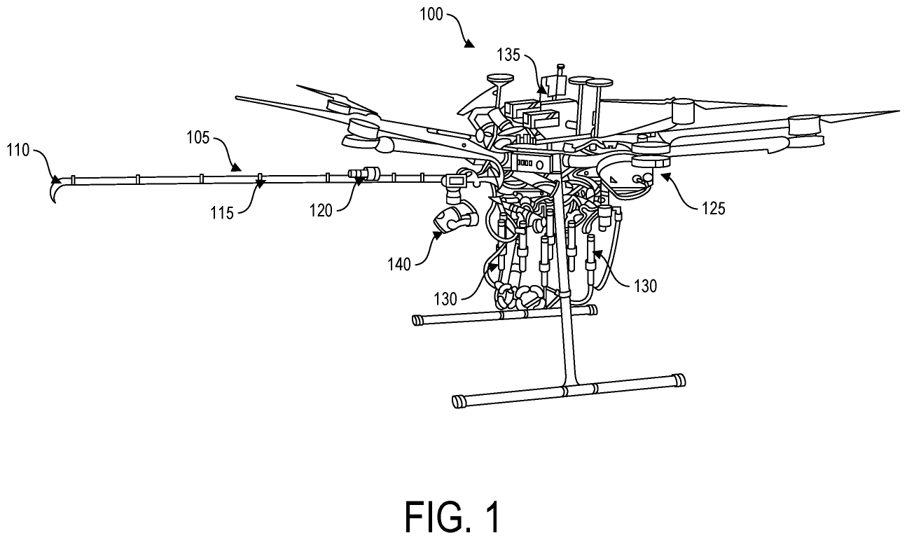

FIG. 1 is an image illustrating an exemplary embodiment of an aerial sampling platform according to subject matter described herein. As shown in FIG. 1, an aerial sampling platform 100 is illustrated. The aerial sampling platform 100 can be a UAV with multiple rotor blades, motors, a power supply such as a battery and/or fuel supply, landing gear, one or more computing devices, communications equipment, sensors, and a payload. In some embodiments, the payload can include a gas sampling payload including one or more sensors, a manifold, one or more sample conduits and one or more filter tubes. The payload can also include computing devices, a controller, and control circuitry configured to operate the manifold.

As shown in FIG. 1, the aerial sampling platform 100 includes an extendable boom 105. The boom 105 can extend or retract relative to the body of the platform 100. The boom 105 can convey a sample conduit 115 extending along the length of the boom 105. A sample inlet 110 can be located at the end of the sample conduit 115. A filter 120, such as a filter contained within a filter tube, can be configured in line (e.g., in fluidic communication) with the sample inlet 110. The sample conduit 115 can be coupled to one or more sensors 125. For example, in some implementations, the one or more sensors can include an oxygen sensor, a carbon monoxide sensor, a methane sensor, a hydrogen sulfide sensor, or a VOC sensor. Additionally, in some embodiments, one of the filters 120 can include a benzene filter. For example, as shown in FIG. 1, a benzene filter 130 can be coupled to a benzene sensor 135 via a sample conduit 115.

As further shown in FIG. 1, the aerial sampling platform 100 can include an image sensor 140. In some embodiments, the image sensor 140 can include a camera, an RGB sensor, or an infrared sensor. Image data collected by the image sensor 140 can be combined with sample data to provide image data overlaid with sample data for a given area or geographic location.

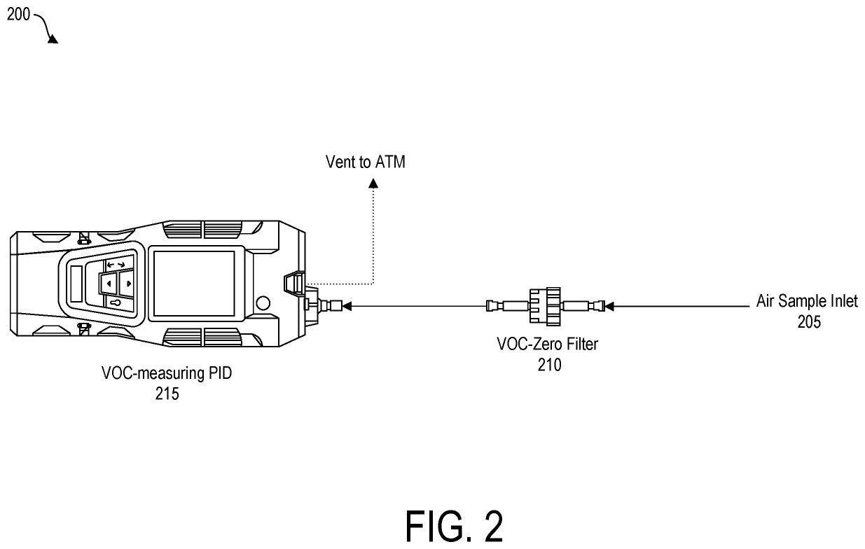

FIG. 2 is a diagram illustrating an exemplary embodiment of a sample conduit of the aerial sampling platform of FIG. 1 according to subject matter described herein. As shown in FIG. 2, a sample conduit can include a sample inlet 205 at which a gas can enter the sample conduit 200. The sample inlet 205 can be fluidically coupled to a filter 210. The filter 210 can correspond to the type of gas being sampled. For example, the filter 210 can include a benzene filter or a VOC filter. The sample conduit 200 can also include a sensor 215. The sensor 215 can be fluidically coupled to the filter 210 so that gas entering the sample inlet 205 is conveyed through the filter 210 to be sensed by the sensor 215. The sensor 215 can vent to the atmosphere. In some embodiments, the sensor 215 can include an oxygen sensor, a carbon monoxide sensor, a methane sensor, a hydrogen sulfide sensor, a benzene sensor, or a VOC sensor.

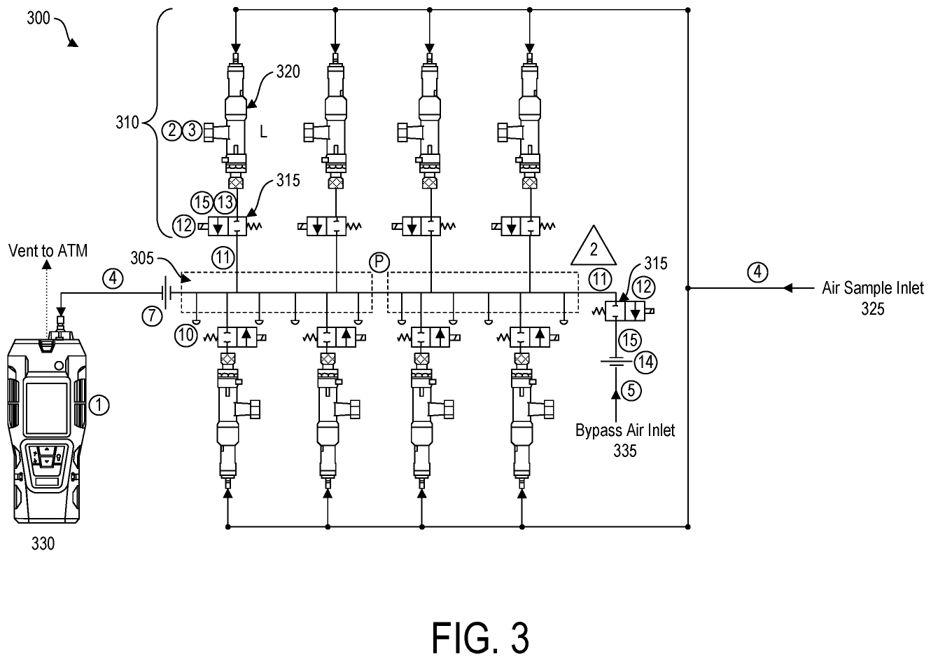

FIG. 3 is a diagram illustrating an exemplary embodiment of a sampling manifold of the aerial sampling platform of FIG. 1 according to subject matter described herein. As shown in FIG. 3, a manifold 305 can be fluidically coupled to one or more sample conduits 310. Each sample conduit 310 can include a valve 315 coupled to a corresponding filter tube 320. Each filter tube 320 can include a filter therein. The filter can be coupled to a sample inlet 325.

The manifold 305 can be coupled to 2, 4, 6, 8, or 10 sample conduits 310, although more or less sample conduits 310 can also be coupled to the manifold 305. The manifold 305 can control delivery of a sample of gas to the sensor 330. Each of the valves 315 can be controlled via control signals generated by a controller to control opening and closing of the valve. In this way, one or more sensors 330 coupled a manifold 305 can each receive different gas samples. The manifold 305 can also be coupled to a valve 315 configured to receive air from a bypass air inlet 335.

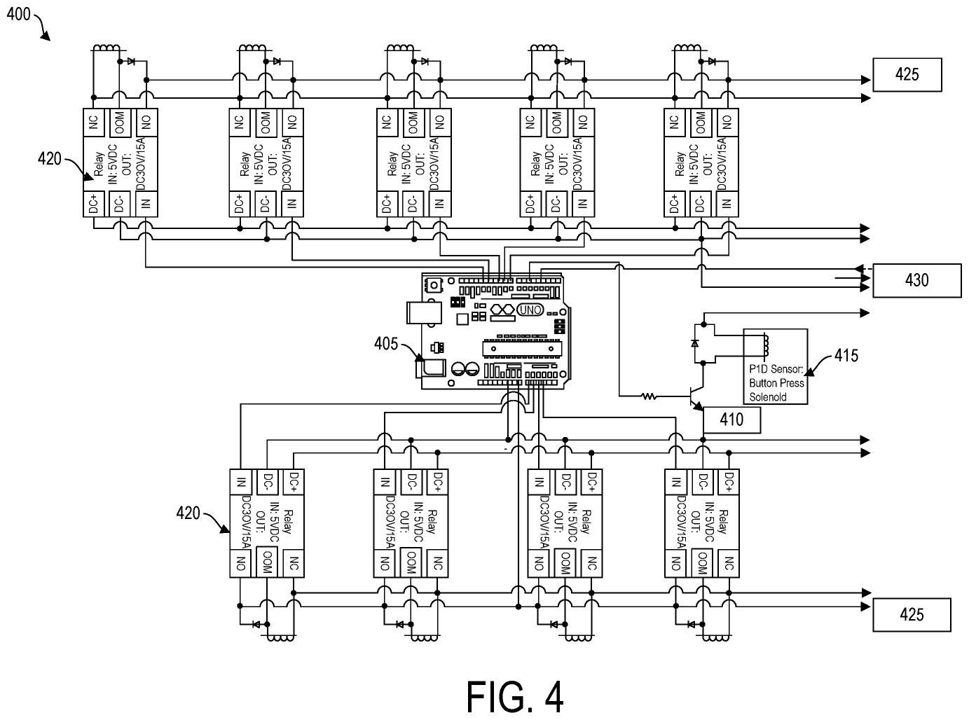

FIG. 4 is a diagram illustrating an exemplary embodiment of control circuitry of the aerial sampling platform of FIG. 1 according to subject matter described herein. As shown in FIG. 4, the circuitry 400 can include a controller 405 coupled to a computing device 410 and a sensor 415. The computing device 410 can include a memory storing computer-readable, executable instructions, which when executed by a processor of the computing device 410 can perform operations of the aerial sampling platform described herein. The sensor 415 can include any one of the sensors described herein.

The circuitry 400 can also include one or more relays 420. Each relay 420 can couple to a valve 425, such as the valve 315 described in relation to the manifold 305 of FIG. 3. The relay 420 can receive control data from the controller 405 to control opening or closing of the valve 425. The circuitry 400 can perform operations of controlling the valves 425 so as to selectively sample gases via the sample conduits coupling a sample inlet with a sensor of the aerial sampling platform. A variety of control programs or operations can be performed to sequentially collect gas samples in a pre-determined way based on real-time sensor data. In some embodiments, the control programs can cause gas samples to be collected in response to operator inputs based on real-time sensor data provided to the operator.

In some embodiments, the aerial sampling platform can include one or more control programs configured for long duration sampling. For example, the aerial sampling platform can include control programs that can operate the valves connected to the pre-filters in a sequential “make-before-break” fashion. This can allow a sample to be collected for a length of time that would exceed a collection time associated with a single pre-filter. This longer sampling period can allow for a larger amount of continuous data to be collected. Sampling can be triggered manually by an operator of the aerial sampling platform or automatically via pre-programmed flight patterns or other automated control programs/logic.

In some embodiments, the aerial sampling platform can include one or more control programs configured for single-filter or shorter-duration sampling. For example, the aerial sampling platform can include control programs that can operate the valves connected to the pre-filters for a duration that is shorter than or equal to the sample time associated with a single pre-filter. The control program can track the sample time of each filter and can adjust the control valves accordingly so that valves are changed to pre-filters before the filter medium is depleted. A gas-sampling pump can be turned on/off on demand and the sampling duration can be changed either manually by the operator of the aerial sampling platform or automatically via automated control programs/logic.

In some embodiments, samples can be collected for milliseconds to minutes. A filter may only be configured for use for a specific sample period. The controller 405 can be configured to optimize sample times based on the type of filter being used, the type of gas to be sensed, or a specific experimental protocol of various sample times, sample gases (or sample filters), or combinations thereof.

As shown in FIG. 4, in some embodiments, one of the relays 420 can be coupled to an input 430. Input 430 is an input from the aerial sampling platform's flight controller to a payload microcontroller. Input 430 can be triggered either manually by the operator of the aerial sampling platform or automatically by payload actions programmed during the flight planning process or through another automated control programs/logic. For example, in one embodiment, a site can require 8 inspection points to be visited at specific coordinates. The aerial sampling platform can automatically fly to each GPS point and can then trigger to the payload to begin gas sampling. In another embodiment, the aerial sampling platform can be used manually maneuvered during an inspection. The operator of the aerial sampling platform can fly to a point-of-interest and can then presses a payload-action button from a remote control which can translate into a trigger signal being passed from the flight controller to the payload microcontroller via input 430.

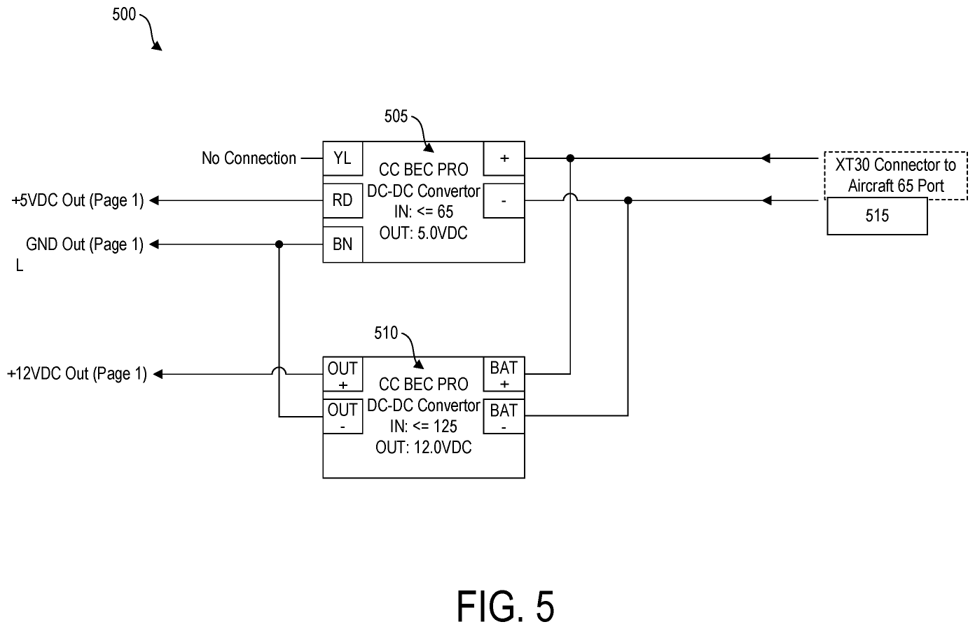

FIG. 5 is an diagram illustrating an exemplary embodiment of a power adapter circuit of the aerial sampling platform of FIG. 1 according to subject matter described herein. The power adapter circuit can transform power from aircraft batteries or power supplies to consistent voltages required by payload electronics configured on the aerial sampling platform of FIG. 1. As shown in FIG. 5, the circuit 500 can include a 5V power adapter 505 and a 12V power adapter 510. The power adapters 505 and 510 can be coupled to a power source 515 of the aerial sampling platform. Power adapters 505 and 510 can provide signal conditioning so that constant-voltage devices can be run from a non-constant voltage source. One or more power adapters can be included and may not be limited to 5V and 12V power levels. In some embodiments, payload devices can be operated using battery power, and therefor would not require any power adapters.

The aerial sampling platform power source 515 can include LiPo batteries in some embodiments, but other battery types or fuel sources can be included. The payload, such as the sensors described in relation to FIGS. 1-3, the manifold 305, valves 315, and navigation systems can be powered by the power source 515 or they can have their own power sources that are separate from the power source 515 of the aerial sampling platform of FIG. 1.

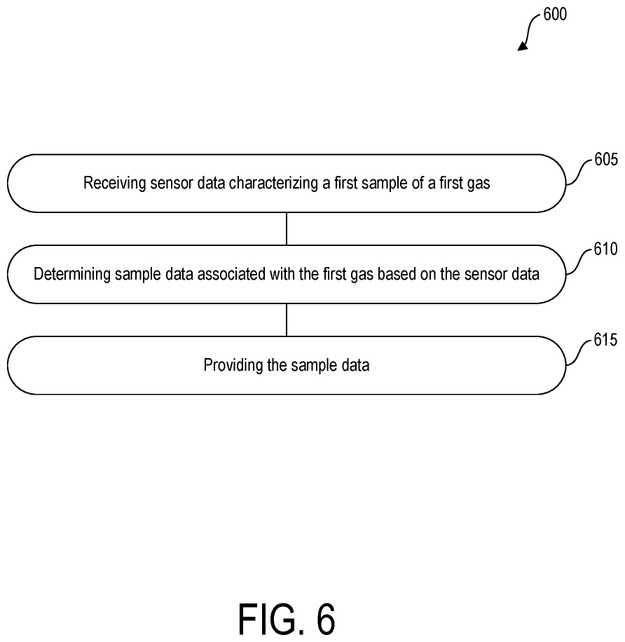

FIG. 6 is a process flow diagram for determining and providing sample data of a sample collected by the aerial sampling platform of FIG. 1 according to subject matter described herein. As shown in FIG. 6, at 605, the process 600 includes receiving sensor data characterizing a first sample of a first gas. The sensor data can be generated based on the sample of gas received via the sample inlet and provided to the gas sensor via the sample conduit of the manifold that is fluidically attached to the gas sensor. The sensor can determine sensor data based on samples of gas received. The sensor data can be provided to a processor of a computing device configured on the aerial sampling platform. In some embodiments, the sensor data can be received based on control signals provided to a controller operable to open or close a valve of the sample conduit. For example, in response to a control signal to open the valve, a sample of gas can be provided to the gas sensor via the sample conduit and sensor data associated with the gas can be generated.

In some embodiments, coordinate data corresponding to a location at which the sample data was obtained can also be received. The coordinate data can include GPS coordinate data.

At 610, the processor can determine sample data associated with the gas based on the sensor data received at 605. The sample data can include a concentration of the gas and a type of gas. In some embodiments, the sensor can determine sample data, such as a concentration of the gas or a type of the gas. In some embodiments, the sample data can be compared to threshold data to determine if an alert should be generated in regard to the sample data of the gas. In some embodiments, the sensor data and/or the sample data can be processed with image data acquired via an image sensor of the aerial sampling platform. The image data can correspond to a location at which the gas was sampled.

At 615, the processor can provide the sample data. The sample data can be stored in a memory of the computing device on the aerial sampling platform or can be transmitted via the communications interface to a second computing device where the sample data can be stored or provided to an operator.

The subject matter described herein provides many technical advantages. For example, some implementations of the aerial sampling platform described herein can provide more efficient sampling of hazardous gases by way of controlling sample conduit valves of a manifold on a UAV platform to increase sample quantities while reducing sample collection times. Some implementations of the aerial sampling platform can improve control of sampling methods in hazardous environments by reducing exposure time for human operators in hazardous emission areas and collecting samples more precisely via the manifold valve operation by the controller. Embodiments, of the aerial sampling platform described herein can provide sample or sensor data with location data so as to output geo-located mapping data of gas emissions. Some implementations of the aerial sampling platform described herein can provide improved control or sample data interfaces for visualizing gas emissions and controlling the aerial sampling platform in regard to geographic locations.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions in a non-transitory way, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system comprising:
an unmanned aerial vehicle (UAV) including
at least one gas sensor;
a manifold coupled to the at least one gas sensor, the manifold including at least one sample conduit comprising a sample inlet, a filter, and a valve coupled to the filter;
a controller; and
a computing device coupled to the controller, the computing device including a processor, a memory, and a communications interface, wherein the processor is configured to perform operations comprising
receiving sensor data characterizing a first sample of a first gas sampled via the at least one gas sensor;
determining sample data associated with the first gas based on the sensor data, the sample data including a concentration and a type of the first gas; and
providing the sample data.

2. The system of claim 1, wherein the controller is configured to control opening and closing of the valve based on a predetermined control sequence or operator inputs provided via the communications interface.

3. The system of claim 2, wherein the sensor data is received in real-time.

4. The system of claim 1, wherein the manifold includes 2, 4, 6, or 8 sample conduits.

5. The system of claim 1, further comprising an image sensor.

6. The system of claim 5, wherein providing the sample data further comprises overlaying the sensor data and/or sample data atop image data acquired via the image sensor.

7. The system of claim 1, wherein providing the sample data includes transmitting the sensor data and/or the sample data to a second computing device via the communications interface.

8. The system of claim 1, wherein providing the sample data further comprises providing GPS coordinate data with the sample data.

9. The system of claim 1, wherein the at least one sensor is a benzene sensor.

10. A method comprising:
receiving sensor data characterizing a first sample of a first gas, the sensor data received by a data processor of a computing device of an unmanned aerial vehicle (UAV) including at least one sensor configured to sample the first gas and generate the sensor data;
determining sample data associated with the first gas based on the sensor data, the sample data including a concentration and a type of the first gas; and
providing the sample data.

11. The method of claim 1, wherein the first gas includes one of oxygen, carbon monoxide, hydrogen sulfide, benzene, methane, or a volatile organic compound.

12. The method of claim 1, wherein the data is received when the UAV is in flight or on the ground.

13. The method of claim 1, wherein the determining and the providing are performed in real-time or near real-time.

14. The method of claim 1, wherein the UAV includes an extended boom and the at least one sensor is configured at a first end of the extended boom.

15. The method of claim 1, wherein the UAV includes a manifold coupled to a plurality of filter tubes, at least one filter tube included in a sample conduit coupling the at least one sensor and to a sample inlet via the manifold.

16. The method of claim 15, wherein the sample conduit includes a valve operable in response to control signals received from a controller of the UAV, the controller configured to open the valve allowing the first gas to be sampled by the at least one sensor.

17. The method of claim 10, further comprising navigating the UAV to a point of interest prior to sampling the first gas.

18. The method of claim 10, further comprising comparing the sample data to threshold data corresponding to the concentration of the gas.

19. The method of claim 18, further comprising generating an alert based on the comparison.

20. The method of claim 1, further comprising transmitting the sample data to a second computing device communicably coupled to the computing device of the UAV.
